SEQUENCE LISTING (4 sequences)

SEQ ID NO: 1 - DNA, 265 bp, Mus Musculus
  misc_feature (0)...(0): Isolate P815 Mouse Mastocytoma cell line, strain DBA, ATCC deposit no. TIB-64
tacacatcgagtcccggaaatcaaagcaaagaaattcagaatttgagatatttgttgactgcgacatcagccgagaacagttgaatgacatcttccccctgctgaagtcgcacgccaccgtcctctcggtggactcgcccgatcagctcactgcgaaggaagacgttatggagactgtcccttggtttccaaagaagatttctgacctggacttctgcgccaacagagtgctgttgtatggatccgaacttgacgccgaccaccc

SEQ ID NO: 2 - DNA, 24 bp, Artificial Sequence
  Primer, MTPH-874F
cacagttcagatcccctctacact

SEQ ID NO: 3 - DNA, 20 bp, Artificial Sequence
  Primer, MTPH-962R
gcaaaactgggttcagccaa

SEQ ID NO: 4 - DNA, 27 bp, Artificial Sequence
  MTPH-926T
aggagttcatggcaggtgtctggctct

(19) United States
(12) Patent Application Publication
(10) Pub. No.: US 2006/0183744 A1
(43) Pub. Date: Aug. 17, 2006

(54) Method for treating depression and/or anxiety

(76) Inventors: Susan P. Rohrer, Scotch Plains, NJ (US); Milton L Hammond, Branchburg, NJ (US)

Correspondence Address: MERCK AND CO., INC, P O BOX 2000, RAHWAY, NJ, 07065-0907, US

(21) Appl. No.: 10/528,212

(22) PCT Filed: Sep. 15, 2003
(86) PCT No.: PCT/US03/29068

§ 371 Date: Mar. 17, 2005

Related U.S. Application Data
(60) Provisional application No. 60/411,919, filed on Sep. 19, 2002.

(51) Int. Cl. A61K 31/50; A61K 38/22; A61K 31/137; A61K 31/59; A61K 31/496

(52) U.S. Cl. 514/247; 514/649; 514/253.03; 514/253.04; 514/167; 514/2; 514/171

(57) ABSTRACT

This invention relates to the treatment and/or prevention of depression and/or anxiety disorders and/or dementia by the administration of an estrogen receptor beta (ERβ) selective agonist either as a single agent, or in combination with other agents.

### BACKGROUND OF THE INVENTION

This invention relates to the treatment and/or prevention of depression and/or anxiety disorders and/or dementia by the administration of an estrogen receptor beta (ERβ) selective agonist either as a single agent, or in combination with other agents.

A major depressive episode has been defined as being a period of at least two weeks during which, for most of the day and nearly every day, there is either depressed mood or the loss of interest or pleasure in all, or nearly all activities. The individual may also experience changes in appetite or weight, sleep and psychomotor activity; decreased energy; feelings of worthlessness or guilt; difficulty thinking, concentrating or making decisions; and recurrent thoughts of death or suicidal ideation, plans or attempts. One or more major depressive episodes may give rise to a diagnosis of major depressive disorder (Diagnostic and Statistical Manual of Mental Disorders, Fourth Edition, American Psychiatric Association, 1994).

Treatment regimens commonly include the use of tricyclic antidepressants (TCAs), monoamine oxidase inhibitors (MAOIs), some psychotropic drugs, lithium carbonate, and electroconvulsive therapy (ECT) (see R. J. Baldessarini in Goodman & Gilman's The Pharmacological Basis of Therapeutics, 9th Edition, Chapter 19, McGraw-Hill, 1996 for a review). More recently, new classes of antidepressant drugs are being developed including selective serotonin reuptake inhibitors (SSRIs), specific monoamine reuptake inhibitors and 5-HT_{1A} receptor agonists and antagonists.

The most established drug treatment for the management of depressive illness are the tricyclic antidepressants. For instance, depressed patients with prominent sleep disturbance and anxiety may be treated with a sedating tricyclic antidepressant such as amitriptyline; for other patients, less sedating compounds such as imipramine or desipramine can be used. As well as inhibiting the uptake of noradrenaline and 5-hydroxytryptamine, tricyclic antidepressants also possess antagonist properties at a variety of neurotransmitter receptors, including muscarinic cholinergic receptors, α₁-adrenoceptors and H₁-histamine receptors. These receptor antagonist effects account for much of the side-effect profile of the tricyclic antidepressants, and in particular, their anticholinergic side-effects which are particularly troublesome in patients with prostatic enlargement or glaucoma. Other side-effects include dry mouth, tachycardia, difficulty in visual accommodation, constipation, urinary retention, sexual dysfunction, cognitive impairment, postural hypotension, and weight gain.

Monoamine oxidase inhibitors are generally prescribed for patients who have failed to respond to tricyclic antidepressants or electroconvulsive therapy. As with tricyclic antidepressants, there are a number of side-effects associated with the use of MAOIs including dizziness, muscular twitching, insomnia, confusion, mania, tachycardia, postural hypotension, hypertension, dry mouth, blurred vision, impotence, peripheral oedema, hepatocellular damage and leucopenia.

Of the new classes of antidepressant, selective serotonin reuptake inhibitors are increasingly prescribed, particularly in patients where the use of tricyclic antidepressants is contraindicated because of their anticholinergic and cardiotoxic effects. SSRIs such as fluoxetine, fluvoxamine, sertraline and paroxetine are generally non-sedating. Furthermore, SSRIs do not stimulate appetite and may therefore be appropriate in patients in whom weight gain would be undesirable. However, SSRIs are not without their own side-effects, including nausea, diarrhea, dry mouth, reduced appetite, dyspepsia, vomiting, headache, nervousness, insomnia, anxiety, tremor, dizziness, fatigue, decreased libido, pharyngitis, dyspnoea, skin rash and sexual dysfunction.

Whatever drug is used, there is a delay of usually two, three or even four weeks before a therapeutic effect is observed. This period of delay may be particularly difficult for a patient suffering from a major depressive illness.

Anxiety is an emotional condition characterized by feelings such as apprehension and fear accompanied by physical symptoms such as tachycardia, increased respiration, sweating and tremor. It is a normal emotion but when it is severe and disabling it becomes pathological.

Anxiety disorders are generally treated using benzodiazepine sedative-antianxiety agents. Potent benzodiazepines are effective in panic disorder as well as in generalized anxiety disorder, however, the risks associated with the drug dependency may limit their long-term use, 5-H1A receptor partial agonists also have useful anxiolytic and other pyschotropic activity, and less likelihood of sedation and dependence (See, e.g., R. J. Balderssarini in Goodman & Gilman's The Pharmacological Basis of Therapeutics, 9^{th} Edition, Chapter 18, McGraw-Hill, 1996, for a review).

Dementia is a syndrome due to disease of the brain, usually a chronic or progressive nature, in which there is a disturbance of multiple higher cortical functions, including memory, thinking, orientation, comprehension, calculation, learning capacity, language and judgement. Consciousness is not clouded. Impairments of cognitive functioning are commonly accompanied, and occasionally preceded, by deterioration in emotional control, social behavior or motivation. This syndrome occurs in Alzheimer's disease, in cerebrovascular disease, and in other conditions primarily or secondarily affecting the brain.

In view of the short-comings of existing antidepressant therapy, there is a need for new, safe and effective treatment for depression and anxiety.

Clinical studies have demonstrated the efficacy of the natural estrogen, 17β-estradiol for the treatment of various forms of depressive illness (See, e.g., Schmidt P J, Nieman L, Danaceau M A, Tobin M B, Roca C A, Murphy J H, Rubinow D R, “Estrogen replacement in perimenopause-related depression: a preliminary report.” Am J Obstet Gynecol 183:414-20, 2000 and Soares C N, Almeida O P, Joffe H, Cohen L S, “Efficacy of estradiol for the treatment of depressive disorders in perimenopausal women: a double-blind, randomized, placebo-controlled trial.” Arch Gen Psychiatry 58:537-8, 2001. It has also been suggested that the anti-depressant activity of estrogen may be mediated via regulation of tryptophan hydroxylase activity and subsequently serotonin synthesis (See, e.g., Lu N Z, Shlaes T A, Cundlah C, Dziennis S E, Lyle R E, Bethea C L, “Ovarian steroid action on tryptophan hydroxylase protein and serotonin compared to localization of ovarian steroid receptors in midbrain of guinea pigs.” Endocrine 11:257-267, 1999).

The potential value of estrogen as an antidepressant agent has been evaluated in small clinical trials of short duration, however, the pleiotropic nature of estrogen preclude its widespread, more chronic use due to the increased risk of proliferative effects on breast, uterine and ovarian tissues. The physiological responses to estrogen are generally mediated via a series of biochemical events initiated by a selective, high affinity interaction between estrogen and an estrogen receptor. There are two estrogen receptors, ERα and ERβ. The identification of a second estrogen receptor, ERβ, has provided a means by which to identify more selective estrogen agents which have the desired anti-depressant and anxiolytic activity in the absence of the proliferative effects which are mediated by ERα. We have demonstrated the co-localization of ERβ (and not ERα) in the serotonin containing cells of the rodent raphe nucleus. Using ERβ selective compounds, estrogen increases transcription of the tryptophan hydroxylase (TPH, the key enzyme in serotonin synthesis) gene via an ERβ mediated event. Thus, the use of ERβ selective agonists can be useful in increasing anti-depressant activity.

### SUMMARY OF THE INVENTION

The present invention relates to the use of an ERβ selective agonist for treating the following disorders in a mammal: depression, perimenopausal depression, post-partum depression, premenstrual syndrome, manic depression, anxiety, dementia, obsessive compulsive behavior, mild cognitive impairment, attention deficit disorder, sleep disorders, irritability, impulsivity, anger management, multiple sclerosis and Parkinsons disease in a mammal, preferably a human. Accordingly, the present invention provides a method for treating the above mentioned disorders in a mammal comprising the administration of ERβ selective agonist.

### DETAILED DESCRIPTION OF THE INVENTION

The present invention relates to the use of an ERβ selective agonist for treating depression in a mammal. The present invention also relates to the use of an ERβ selective agonist for treating anxiety in a mammal. The present invention also relates to the use of an ERβ selective agonist for treating dementia in a mammal.

Illustrating the invention is the use of a CNS penetrant ERβ selective agonist for the treatment of depression, perimenopausal depression, post-partum depression, manic depression, anxiety, dementia and/or obsessive compulsive behavior.

Further illustrating the invention is the use of an orally active ERβ selective agonist for the treatment of depression, perimenopausal depression, post-partum depression, manic depression, anxiety, dementia and/or obsessive compulsive behavior.

Illustrating the invention is the use of a CNS penetrant, orally active ERβ selective agonist for the treatment of mild congnitive impairment, attention disorders, sleep disorders, irritability, impulsivity and anger.

Also, illustrating the invention is the use of a CNS penetrant, orally active ERβ selective agonist for the treatment of multiple sclerosis, Parkinson's disease, chronic-pain, rheumatoid arthritis, inflammatory bowel disease, irritable bowel disease, prostate hyperplasia and prostate cancer.

Also illustrating the invention is the use of an ERβ selective agonist for the treatment of major depressive disorder.

Exemplifying the invention is the use of an ERβ selective agonist for the treatment of depression including depressive disorders, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder; perimenopausal depression; postpartum depression.

Further exemplifying the invention is the use of an ERβ selective agonist for the treatment of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalized anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delirium, dementia, and amnestic and other cognitive or neurodegenerative disorders, such as Alzheimer's disease, senile dementia, dementia of the Alzheimer's type, vascular dementia, and other dementias, for example, due to HIV disease, head trauma, Parkinson's disease, Huntington's disease, Pick's disease, Creutzfeldt-Jakob disease, or due to multiple aetiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor; substance-related disorders arising from the use of alcohol, amphetamines (or amphetamine-like substances) caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delirium, withdrawal delirium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid hemorrhage or cerebral oedema.

The ERβ selective agonists of use in the present invention may be any ERβ selective agonist known from the art.

The ERβ selective agonist may be steroidal or non-steroidal in nature. Use of an orally active ERβ selective agonist is preferred.

In the present invention, it is preferred that the ERβ selective agonist active upon the central nervous system (CNS), such as the brain, following systemic administration, i.e. that it readily penetrates the CNS. Accordingly, a preferred ERβ selective agonist for use in the present invention is a CNS-penetrating ERβ selective agonist.

Non-limiting examples of ERβ selective agonists include compounds described in International Publication WO 01/82923, which is hereby incorporated by reference, of the formula: 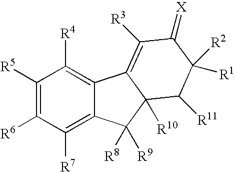
wherein X is selected from the group consisting of: O,N—OR^{a}, N—NR^{a}R^{b} and C₁₋₆ alkylidene,
   wherein said alkylidene group is unsubstituted or substituted with a group selected from hydroxy, amino, O(C₁₋₄alkyl), NH(C₁₋₄alkyl), or N(C₁₋₄alkyl)₂;
R¹ is selected from the group consisting of hydrogen, C₁₋₆alkyl, C₂₋₆alkenyl, and C₂₋₆alkynyl,
   wherein said alkyl, alkenyl and alkynyl groups are either unsubstituted or substituted with a group selected from OR^{c}, SR^{c}, NR^{b}R^{c}, C(═O)R^{c}, C(═O)CH₂OH, or phenyl, wherein said phenyl group can either be unsubstituted
   or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, C₂H, CO₂(C₁₋₄alkyl), C(O)H, and C(O)(C₁₋₄alkyl);
R² is selected from the group consisting of hydrogen, hydroxy, iodo, O(C═O)R^{c}, C(═O)R^{c},
   CO₂R^{c}, C₁₋₆alkyl, C₂₋₆alkenyl, and C₂₋₆alkynyl, wherein said alkyl, alkenyl and alkynyl groups are either unsubstituted or substituted with a group selected from OR^{c}, SR^{c}, NR^{b}R^{c}, C(═O)R^{c}, C(═O)CH₂OH, or phenyl, wherein said phenyl
   group can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
   or R¹ and R², when taken together with the carbon atom to which they are attached, form a carbonyl group;
   or R¹ and R², when taken together, form a C₁₋₆ alkylidene group, wherein said alkylidene group is either unsubstituted or substituted with a group selected from the group consisting of hydroxy, O(C₁₋₄alkyl), N(C₁₋₄alkyl)₂, and phenyl,
   wherein said phenyl group can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
R³ is selected from the group consisting of hydrogen, fluoro, chloro, bromo, iodo, cyano, NR^{a}R^{c}, OR^{a}, C(═O)R^{a}, CO₂R^{c}, CONR^{a}R^{c}, SR^{a}, S(═O)R^{a}, SO₂R^{a}, C₁₋₁₀alkyl, C₂₋₁₀alkenyl, C₂₋₁₀alkynyl, C₃₋₇cycloalkyl, 4-7 membered heterocycloalkyl, cycloalkylalkyl, aryl, heteroaryl, arylalkyl, and heteroarylalkyl, wherein said alkyl, alkenyl, alkynyl, cycloalkyl, aryl and heteroaryl groups are either unsubstituted or independently substituted with 1, 2 or 3 groups selected from fluoro, chloro, bromo, iodo, cyano, OR^{a}, NR^{a}R^{c}, O(C═O)R^{a}, O(C═O)NR^{a}R^{c}, NR^{a}(C═O)R^{c}, NR^{a}(C═O)OR^{c}, C(═O)R^{a}, CO₂R^{a}, CONR^{a}R^{c}, CSNR^{a}R^{c}, SR^{a}, S(O)R^{a}, SO₂R^{a}, SO₂NR^{a}R^{c}, YR^{d}, and ZYR^{d};
R⁴ is selected from the group consisting of hydrogen, hydroxy, amino, methyl, CF₃, fluoro, chloro, and bromo;
R⁵ and R⁶ are each independently selected from the group consisting of hydrogen, fluoro, chloro, bromo, methyl, amino, OR^{b}, OR^{a}, O(C═O)R^{c}, O(C═O)OR^{c}, and NH(C═O)R^{c};
R⁷ is selected from the group consisting of hydrogen, OR^{b}, NR^{b}R^{c}, fluoro, chloro, bromo, iodo, cyano, nitro, C₁₋₆alkyl, C₂₋₆alkenyl, CF₃, and CHF₂;
R⁸ and R⁹ are each independently selected from the group consisting of hydrogen, C₁₋₆alkyl, C₂₋₆alkenyl, and C₂₋₆alkynyl,
   or R⁸ and R⁹, when taken together with the carbon atom to which they are attached, form a 3-5 membered cycloalkyl ring,
   or R⁸ and R⁹, when taken together with the carbon atom to which they are attached, form a carbonyl group;
R¹⁰ is selected from the group consisting of hydrogen, C₁₋₁₀alkyl, C₂₋₁₀alkenyl, C₂₋₁₀alkynyl, C₃₋₆cycloalkyl, cycloalkylalkyl, aryl, heteroaryl, arylalkyl and heteroarylalkyl,
   wherein said alkyl, alkenyl, alkynyl, cycloalkyl, cycloalkylalkyl, aryl, heteroaryl, arylalkyl and heteroarylalkyl groups can be optionally substituted with a group selected from chloro, bromo, iodo, OR^{b}, SR^{b}, C(═O)R^{b}, or 1-5 fluoro, or R¹⁰ and R¹, when taken together with the three intervening carbon atoms to which they are attached, form a 5-6 membered cycloalkyl or cycloalkenyl ring which can be optionally substituted with 1 or 2 groups selected from oxo, hydroxy, or C₁₋₆alkyl;
R¹¹ is selected from the group consisting of hydrogen and C₁₋₄alkyl;
R^{a} is selected from the group consisting of hydrogen, C₁₋₁₀alkyl, and phenyl, wherein said alkyl group can be optionally substituted with a group selected from hydroxy, amino, O(C₁₋₄alkyl), NH(C₁₋₄alkyl), N(C₁₋₄alkyl)₂, phenyl, or 1-5 fluoro, and wherein said phenyl groups can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
R^{b} is selected from the group consisting of hydrogen, C₁₋₁₀alkyl, benzyl and phenyl, wherein said phenyl group can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
R^{c} is selected from the group consisting of hydrogen, C₁₋₁₀alkyl and phenyl, wherein said phenyl group can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
   or R^{a} and R^{c}, whether or not on the same atom, can be taken together with any attached and intervening atoms to form a 4-7 membered ring;
R^{d} is selected from the group consisting of NR^{b}R^{c}, OR^{a}, CO₂R^{a}, O(C═O)R^{a}, CN, NR^{c}(C═O)R^{b}, CONR^{a}R^{c}, SO₂NR^{a}R^{c}, and a 4-7 membered N-heterocycloalkyl ring that can be optionally interrupted by O, S, NR^{c}, or C═O;
Y is selected from the group consisting of CR^{b}R^{c}, C₂₋₆alkylene and C₂₋₆alkenylene, wherein said alkylene and alkenylene linkers can be optionally interrupted by O, S, or NR^{c};
Z is selected from the group consisting of O, S, NR^{c}, C═O, O(C═O), (C═O)O, NR^{c}(C═O) or (C═O)NR^{c};
   and the pharmaceutically acceptable salts thereof.

Non-limiting examples of ERβ selective agonists further include compounds of the formula: 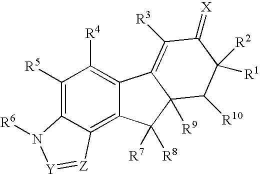
wherein X is selected from the group consisting of: O, N—OR^{a}, N—NR^{a}R^{b} and C₁₋₆ alkylidene, wherein said alkylidene group is unsubstituted or substituted with a group selected from hydroxy, amino, O(C₁₋₄alkyl), NH(C₁₋₄alkyl), or N(C₁₋₄alkyl)₂;
Y is selected from the group consisting of N and CR^{e};
Z is selected from the group consisting of N and CR^{f};
R¹ is selected from the group consisting of hydrogen, C₁₋₆alkyl, C₂₋₆alkenyl, and C₂₋₆alkynyl, wherein said alkyl, alkenyl and alkynyl groups are either unsubstituted or substituted with a group selected from OR^{c}, SR^{c}, NR^{b}R^{c}, C(═O)R^{c}, C(═O)CH₂OH, bromo, 1-3 chloro, 1-5 fluoro or phenyl, wherein said phenyl group can either be unsubstituted or substituted with a substituent selected from the group consisting of C₁₋₄alkyl, OH and O(C₁₋₄alkyl);
R² is selected from the group consisting of hydrogen, hydroxy, iodo, O(C═O)R^{c}, C(═O)R^{c}, CO₂R^{c}, C₁₋₆alkyl, C₂₋₆alkenyl, and C₂₋₆alkynyl, wherein said alkyl, alkenyl and alkynyl groups are either unsubstituted or substituted with a group selected from OR^{c}, SR^{c}, NR^{b}R^{c}, C(═O)R^{c}, C(═O)CH₂OH, or phenyl, wherein said phenyl group can either be unsubstituted or substituted with a substituent selected from the group consisting of C₁₋₄alkyl, OH and O(C₁₋₄alkyl);
   or R¹ and R², when taken together with the carbon atom to which they are attached, form a carbonyl group;
   or R¹ and R², when taken together with the carbon atom to which they are attached, form a C₃₋₇cycloalkyl or 3-7 heterocycloalkyl ring, wherein said ring is either unsubstituted or substituted with a group selected from C₁₋₄alkyl, OH, O(C₁₋₄alkyl) and oxo;
   or R¹ and R², when taken together, form a C₁₋₆alkylidene group, wherein said alkylidene group is either unsubstituted or substituted with a group selected from the group consisting of hydroxy, O(C₁₋₄alkyl), N(C₁₋₄alkyl)₂, and phenyl, wherein said phenyl group can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)2, halo, CN, NO₂, CO₂H, C)₂(C₁₋₄alkyl), C(O)H, and C(O)(C₁₋₄alkyl);
R³ is selected from the group consisting of hydrogen, fluoro, chloro, bromo, iodo, cyano, nitro, NR^{a}R^{c}, OR^{a}, S(O)R^{a}, SO₂R^{a}, SR^{a}, C(═O)R^{a}, CO₂R^{c}, CONR^{a}R^{c}, C₁₋₁₀alkyl, C₂₋₁₀alkenyl, C₂₋₁₀alkynyl, C₃₋₇cycloalkyl, 4-7 membered heterocycloalkyl, cycloalkylalkyl, aryl, heteroaryl, arylalkyl, and heteroarylalkyl, wherein said alkyl, alkenyl, alkynyl, cycloalkyl, aryl and heteroaryl groups are either unsubstituted or independently substituted with 1, 2 or 3 groups selected from fluoro, chloro, bromo, iodo, cyano, OR^{a}, NR^{a}R^{c}, O(C═O)R^{a}, O(C═O)NR^{a}R^{c}, NR^{a}(C═O)R^{c}, NR^{a}(C═O)OR^{c}, C(═O)R^{a}, CO₂R^{a}, CONR^{a}R^{c}, CSNR^{a}R^{c}, SR^{a}, S(O)R^{a}, SO₂R^{a}, SO₂NR^{a}R^{c}, LR^{d}, and MLR^{d};
R⁴ and R⁵ are each independently selected from the group consisting of hydrogen, hydroxy, amino, methyl, CF₃, fluoro, chloro, and bromo;
R⁶ is selected from the group consisting of hydrogen, (C═O)R^{a}, (C═O)OR^{a}, and SO₂R^{a};
R⁷ and R⁸ are each independently selected from the group consisting of hydrogen, C₁₋₆alkyl, C₂₋₆alkenyl, C₂₋₆alkynyl, fluoro, chloro, bromo, cyano, hydroxy, O(C₁₋₆alkyl), azido, amino, NH(C₁₋₄alkyl), and N(C₁₋₄alkyl)₂;
   or R⁷ and R⁸, when taken together with the carbon atom to which they are attached, form a 3-5 membered cycloalkyl ring;
   or R⁷ and R⁸, when taken together with the carbon atom to which they are attached, form a carbonyl group;
   or R⁷ and R⁸, when taken together, form a C₁₋₆alkylidene group, wherein said alkylidene group is either unsubstituted or substituted with a group selected from cyano, C(═O)H, C(═O)(C₁₋₄alkyl), or C(═O)OC₁₋₄alkyl;
R⁹ is selected from the group consisting of hydrogen, C₁₋₁₀alkyl, C₂₋₁₀alkenyl, C₂₋₁₀alkynyl, C₃₋₆cycloalkyl, cycloalkylalkyl, aryl, heteroaryl, arylalkyl and heteroarylalkyl, wherein said alkyl, alkenyl, alkynyl, cycloalkyl, cycloalkylalkyl, aryl, heteroaryl, arylalkyl and heteroarylalkyl groups can be optionally substituted with a group selected from bromo, iodo, OR^{b}, SR^{b}, C(═O)R^{b}, 1-3 chloro, or 1-5 fluoro;
   or R⁹ and R¹, when taken together with the three intervening carbon atoms to which they are attached, form a 5-6 membered cycloalkyl or cycloalkenyl ring which can be optionally substituted with 1-3 groups independently selected from oxo, hydroxy, fluoro, C₁₋₆alkyl, C₂₋₆alkenyl, C₂₋₆alkynyl, C₁₋₆alkylidenyl, C₃₋₆cycloalkyl, cycloalkylalkyl, phenyl, or phenylalkyl, wherein said alkyl, alkenyl, alkyl, alkylidenyl, cycloalkyl, cycloalkylalkyl, phenyl, and phenylalkyl groups can be optionally substituted with a group selected from chloro, bromo, iodo, OR^{b}, SR^{b}, C₁₋₃alkyl, C(═O)R^{b}, or 1-5 fluoro;
   or R⁹ and R⁸, when taken together with the two intervening carbon atoms to which they are attached, form a cyclopropyl ring which can be optionally substituted with 1-2 groups independently selected from C₁₋₆alkyl, C₂₋₆alkenyl, C₂₋₆alkynyl, C₃₋₆cycloalkyl, cycloalkylalkyl, phenyl, or phenylalkyl, wherein said alkyl, alkenyl, alkynyl, cycloalkyl, cycloalkylalkyl, phenyl, and phenylalkyl groups can be optionally substituted with a group selected from chloro, bromo, iodo, OR^{b}, SR^{b}, C₁₋₃alkyl, C(═O)R^{b}, or 1-5 fluoro;
R¹⁰ is selected from the group consisting of hydrogen, C₁₋₁₀alkyl, and C₂₋₁₀alkenyl;
R^{a} is selected from the group consisting of hydrogen, C₁₋₁₀alkyl, and phenyl, wherein said alkyl group can be optionally substituted with a group selected from hydroxy, amino, O(C₁₋₄alkyl), NH(C₁₋₄alkyl), N(C₁₋₄alkyl)₂, phenyl, or 1-5 fluoro, and wherein said phenyl groups can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
R^{b} is selected from the group consisting of hydrogen, C₁₋₁₀alkyl, benzyl and phenyl, wherein said phenyl group can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
R^{c} is selected from the group consisting of hydrogen, C₁₋₁₀alkyl and phenyl, wherein said phenyl group can either be unsubstituted or substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
   or R^{a} and R^{c}, whether or not on the same atom, can be taken together with any attached and intervening atoms to form a 4-7 membered ring;
R^{d} is selected from the group consisting of NR^{b}R^{c}, OR^{a}, CO₂R^{a}, O(C═O)R^{a}, CN, NR^{c}(C═O)R^{b}, CONR^{a}R^{c}, SO₂NR^{a}R^{c}, and a 4-7 membered N-heterocycloalkyl ring that can be optionally interrupted by O, S, NR^{c}, or C═O;
R^{e} is selected from the group consisting of hydrogen, C₁₋₆alkyl, C₂₋₆alkenyl, CF₃, halo, O(C₁₋₄alkyl), NH₂, NH(C₁₋₄alkyl), and N(C₁₋₄alkyl)₂;
R^{f} is selected from the group consisting of hydrogen, C₁₋₆alkyl, C₂₋₆alkenyl, CF₃, halo, O(C₁₋₄alkyl), NO₂, NH₂, NH(C₁₋₄alkyl), and N(C₁₋₄alkyl)₂;
L is selected from the group consisting of CR^{b}R^{c}, C₂₋₆ alkylene and C₂₋₆ alkenylene, wherein said alkylene and alkenylene groups can be optionally interrupted by O, S, or NR^{c};
M is selected from the group consisting of O, S, NR^{c}, C═O, O(C═O), (C═O)O, NR(C═O) or (C═O)NR^{c};
   and the pharmaceutically acceptable salts thereof.

Non-limiting examples of ERβ selective agonists further include: 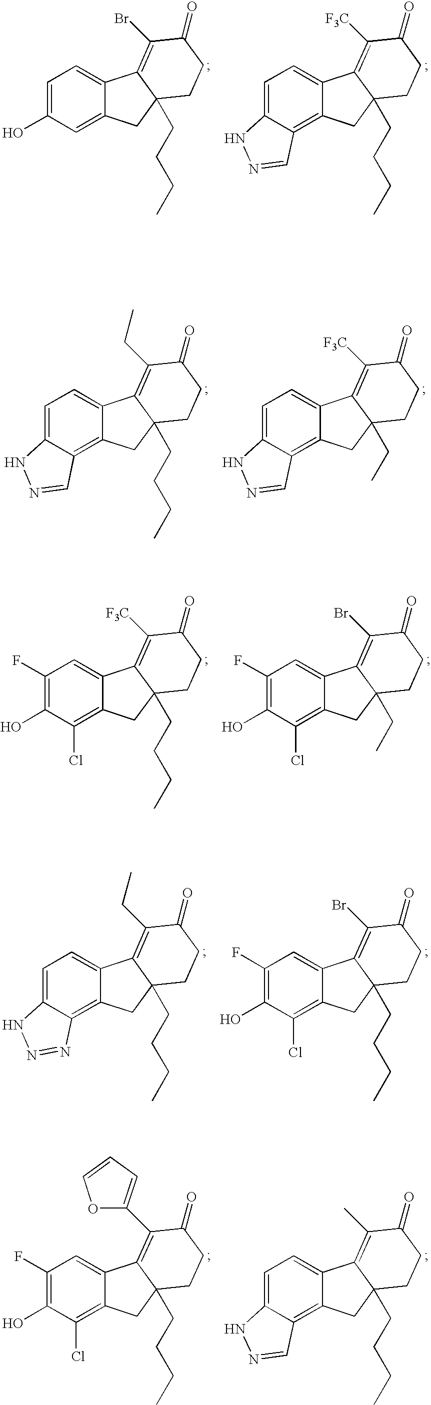 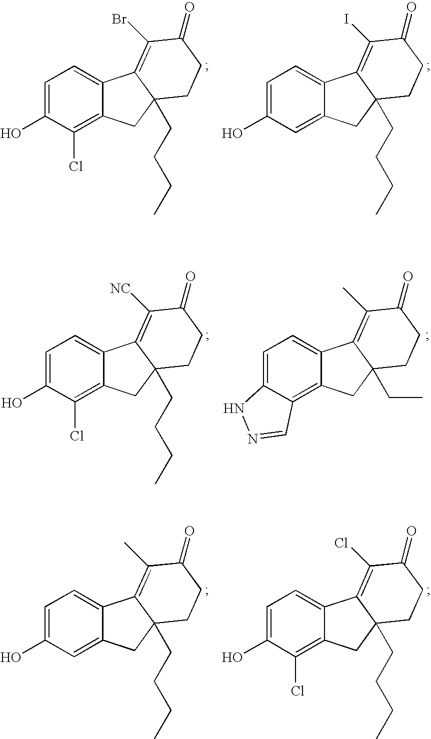
and the pharmaceutically acceptable salts thereof.

The above compounds are only illustrative of ERβ selective agonists. As this listing of groups of compounds is not meant to be comprehensive, the methods of the present invention may employ any ERβ selective agonist and is not limited to any particular structural class of compound.

A suitable selection cascade for ERβ selective agonists of use according to the present invention is as follows:

(i) Determine affinity for human ERβ and ERα in radioligand binding studies (Example 1); select compounds with ERβ IC₅₀≦10 nM, preferably IC₅₀≦2 nM, especially IC₅₀≦1 nM and with selectivity compared to ERα of 20-fold, preferably >50-fold and especially >100-fold; and

(ii) Determine ability of compounds to penetrate CNS by their ability to stimulate tryptophan hydroxylase activity or progesterone receptor expression in the dorsal raphe nucleus of mice treated with the compound either orally or subcutaneously with an EC50<30 mg/kg, and preferably with EC50<10 mg/kg when administered orally. The compound administration is done for 4 days prior to evaluation of efficacy.

The compounds of the present invention may have asymmetric centers, chiral axes, and chiral planes (as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms, and mixtures thereof, are intended to be encompassed by the scope of the invention, even though only one tautomeric structure is depicted.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci, 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Certain of the above defined terms may occur more than once in the above formula and upon such occurrence each term shall be defined independently of the other. Similarly, the use of a particular variable within a noted structural formula is intended to be independent of the use of such variable within a different structural formula.

Full descriptions of the preparation of the ERβ selective agonists which are employed in the present invention may be found in the references cited herein.

The identification of a compound as an ERβ selective agonist, in particular a CNS penetrant ERα selective agonist, and thus able to have utility in the present invention may be readily determined without undue experimentation by methodology well known in the art, such as the assays described herein.

According to a further aspect of the present invention, it may be desirable to treat any of the aforementioned conditions with a combination of an ERα selective agonist and one or more other pharmacologically active agents suitable for the treatment of the specific condition. The ERα selective agonist and the other pharmacologically active agent(s) may be administered to a patient simultaneously, sequentially or in combination. For example, the present compound may be employed directly in combination with the other active agent(s), or it may be administered prior, concurrent or subsequent to the administration of the other active agent(s). In general, the currently available dosage forms of the known therapeutic agents for use in such combinations will be suitable.

Such agents include the following: an organic bisphosphonate; a cathepsin K inhibitor; an estrogen or an estrogen receptor modulator, an androgen receptor modulator; an inhibitor of osteoclast proton ATPase; an integrin receptor antagonist; an osteoblast anabolic agent, such as PTH; calcitonin; Vitamin D or a synthetic Vitamin D analogue; and the pharmaceutically acceptable salts and mixtures thereof. A preferred combination is a compound of the present invention and an organic bisphosphonate. Another preferred combination is a compound of the present invention and a cathepsin K inhibitor. Another preferred combination is a compound of the present invention and an estrogen. Another preferred combination is a compound of the present invention and an androgen receptor modulator. Another preferred combination is a compound of the present invention and an osteoblast anabolic agent

“Organic bisphosphonate” includes, but is not limited to, compounds of the chemical formula 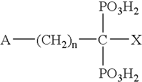
wherein n is an integer from 0 to 7 and wherein A and X are independently selected from the group consisting of H, OH, halogen, NH₂, SH, phenyl, C1-C30 alkyl, C3-C30 branched or cycloalkyl, bicyclic ring structure containing two or three N, C1-C30 substituted alkyl, C1-C10 alkyl substituted NH₂, C3-C10 branched or cycloalkyl substituted NH₂, C1-C10 dialkyl substituted NH₂, C1-C10 alkoxy, C1-C10 alkyl substituted thio, thiophenyl, halophenylthio, C1-C10 alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C3-C10 ring.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazolyl, NH₂, C1-C10 alkyl or dialkyl substituted NH₂, OH, SH, and C1-C10 alkoxy.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C1-30-alkyl-substituted ammonium. Preferred salts are those selected from the group consisting of sodium, potassium, calcium, magnesium, and ammonium salts. More preferred are sodium salts. Non-limiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein. For example, the phrase “about 5 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 5 mg of alendronic acid.

Non-limiting examples of bisphosphonates useful herein include the following:

Alendronic acid, 4-amino-1-hydroxybutylidene-1,1-bisphosphonic acid.

Alendronate (also known as alendronate sodium or alendronate monosodium trihydrate), 4-amino-1-hydroxybutylidene-1,1-bisphosphonic acid monosodium trihydrate.

Alendronic acid and alendronate are described in U.S. Pat. No. 4,922,007, to Kieczykowski et al., issued May 1, 1990; U.S. Pat. No. 5,019,651, to Kieczykowski et al., issued May 28, 1991; U.S. Pat. No. 5,510,517, to Dauer et al., issued Apr. 23, 1996; U.S. Pat. No. 5,648,491, to Dauer et al., issued Jul. 15, 1997, all of which are incorporated by reference herein in their entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

1-hydroxy-2-imidazo-(1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zoledronate).

Nonlimiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

It is recognized that mixtures of two or more of the bisphosphonate actives can be utilized.

The precise dosage of the organic bisphosphonate will vary with the dosing schedule, the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight. For alendronate monosodium trihydrate, common human doses which are administered are generally in the range of about 2 mg/day to about 40 mg/day, preferably about 5 mg/day to about 40 mg/day. In the U.S. presently approved dosages for alendronate monosodium trihydrate are 5 mg/day for preventing osteoporosis, 10 mg/day for treating osteoporosis, and 40 mg/day for treating Paget's disease.

In alternative dosing regimens, the bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of 35 mg/week or 70 mg/week. The bisphosphonates may also be administered monthly, ever six months, yearly or even less frequently, see WO 01/97788 (published Dec. 27, 2001) and WO 01/89494 (published Nov. 29, 2001).

“Estrogen” includes, but is not limited to naturally occurring estrogens [7-estradiol (E₂), estrone (E₁), and estriol (E₃)], synthetic conjugated estrogens, oral contraceptives and sulfated estrogens. See, Gruber C J, Tschugguel W, Schneeberger C, Huber J C., “Production and actions of estrogens” N Engl J Med Jan. 31, 2002;346(5):340-52.

“Estrogen receptor modulators” refers to compounds which interfere or inhibit the binding of estrogen to the receptor, regardless of mechanism. Examples of estrogen receptor modulators include, but are not limited to, estrogen, progestogen, estradiol, droloxifene, raloxifene, lasofoxifene, TSE-424, tamoxifen, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Cathepsin K inhibitors” refers to compounds which interfere with the activity of the cysteine protease cathepsin K. Nonlimiting examples of cathepsin K inhibitors can be found in PCT publications WO 00/55126 to Axys Pharmaceuticals and WO 01/49288 to Merck Frosst Canada & Co. and Axys Pharmaceuticals.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“An inhibitor of osteoclast proton ATPase” refers to an inhibitor of the proton ATPase, which is found on the apical membrane of the osteoclast, and has been reported to play a significant role in the bone resorption process. This proton pump represents an attractive target for the design of inhibitors of bone resorption which are potentially useful for the treatment and prevention of osteoporosis and related metabolic diseases. See C. Farina et al., “Selective inhibitors of the osteoclast vacuolar proton ATPase as novel bone antiresorptive agents,” DDT, 4: 163-172 (1999)), which is hereby incorporated by reference in its entirety.

As used above, “integrin receptor antagonists” refers to compounds which selectively antagonize, inhibit or counteract binding of a physiological ligand to the αᵥβ₃ integrin, to compounds which selectively antagonize, inhibit or counter-act binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins. H. N. Lode and coworkers in PNAS USA 96: 1591-1596 (1999) have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth. αᵥβ₃ integrin receptor antagonists inhibit bone resorption through a new mechanism distinct from that of all currently available drugs. Integrins are heterodimeric transmembrane adhesion receptors that mediate cell-ell and cell-matrix interactions. The α and β integrin subunits interact non-covalently and bind extracellular matrix ligands in a divalent cation-dependent manner. The most abundant integrin on osteoclasts is αᵥβ₃(>10⁷/osteoclast), which appears to play a rate-limiting role in cytoskeletal organization important for cell migration and polarization. The αᵥβ₃ antagonizing effect is selected from inhibition of bone resorption, inhibition of restenosis, inhibition of macular degeneration, inhibition of arthritis, and inhibition of cancer and metastatic growth.

“An osteoblast anabolic agent” refers to agents that build bone, such as PTH. The intermittent administration of parathyroid hormone (H) or its amino-terminal fragments and analogues have been shown to prevent, arrest, partially reverse bone loss and stimulate bone formation in animals and humans. For a discussion refer to D. W. Dempster et al., “Anabolic actions of parathyroid hormone on bone,” Endocr Rev 14: 690-709 (1993). Studies have demonstrated the clinical benefits of parathyroid hormone in stimulating bone formation and thereby increasing bone mass and strength. Results were reported by R M Neer et al., in New Eng J Med 344 1434-1441 (2001).

In addition, parathyroid hormone-related protein fragments or analogues, such as PTHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)] and may also have potential as anabolic agents for treating osteoporosis.

Calcitonin is a 32 amino acid pepetide produced primarily by the thyroid which is known to participate in calcium and phosphorus metabolism Calcitonin suppresses resorption of bone by inhibiting the activity of osteoclasts. Thus, calcitonin can allow osteoblasts to work more effectively and build bone.

“Vitamin D” includes, but is not limited to, vitamin D₃ (cholecalciferol) and vitamin D₂ (ergocalciferol), which are naturally occurring, biologically inactive precursors of the hydroxylated biologically active metabolites of vitamin D: 1α-hydroxy vitamin D; 25-hydroxy vitamin D, and 1α,25-dihydroxy vitamin D. Vitamin D₂ and vitamin D₃ have the same biological efficacy in humans. When either vitamin D₂ or D₃ enters the circulation, it is hydroxylated by cytochrome P₄₅₀-vitamin D-25-hydroxylase to give 25-hydroxy vitamin D. The 25-hydroxy vitamin D metabolite is biologically inert and is further hydroxylated in the kidney by cytochrome P450-monooxygenase, 25 (OH) D-1α-hydroxylase to give 1,25-dihydroxy vitamin D. When serum calcium decreases, there is an increase in the production of parathyroid hormone (PTH), which regulates calcium homeostasis and increases plasma calcium levels by increasing the conversion of 25-hydroxy vitamin D to 1,25-dihydroxy vitamin D.

1,25-dihydroxy vitamin D is thought to be responsible for the effects of vitamin D on calcium and bone metabolism. The 1,25-dihydroxy metabolite is the active hormone required to maintain calcium absorption and skeletal integrity. Calcium homeostasis is maintained by 1,25 dihydroxy vitamin D by inducing monocytic stem cells to differentiate into osteoclasts and by maintaining calcium in the normal range, which results in bone mineralization by the deposition of calcium hydroxyapatite onto the bone surface, see Holick, M F, Vitamin D photobiology, metabolism, and clinical applications, In: DeGroot L, Besser H, Burger H G, eg al., eds. Endocrinology, 3^{rd} ed., 990-1013 (1995). However, elevated levels of 1α,25-dihydroxy vitamin D₃ can result in an increase of calcium concentration in the blood and in the abnormal control of calcium concentration by bone metabolism, resulting in hypercalcemia. 1α, 25-dihydroxy vitamin D₃ also indirectly regulates osteoclastic activity in bone metabolism and elevated levels may be expected to increase excessive bone resorption in osteoporosis.

“Synthetic vitamin D analogues” includes non-naturally occurring compounds that act like vitamin D.

It will be appreciated that for the treatment of depression or anxiety, the ERα selective agonist may be used in conjunction with other anti-depressant or anti-anxiety agents.

Suitable classes of anti-depressant agent include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, melatonin agonists and atypical anti-depressants.

Suitable norepinephrine reuptake inhibitors include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Suitable selective serotonin reuptake inhibitors include: fluoxetine, fluvoxamine, paroxetine and sertraline, and pharmaceutically acceptable salts thereof.

Suitable monoamine oxidase inhibitors include: isocarboxazid, phenelzine, tranylcypromine and selegiline, and pharmaceutically acceptable salts thereof.

Suitable reversible inhibitors of monoamine oxidase include: moclobemide, and pharmaceutically acceptable salts thereof.

Suitable serotonin and noradrenaline reuptake inhibitors of use in the present invention include: venlafaxine, and pharmaceutically acceptable salts thereof.

Suitable CRF antagonists include those compounds described in International Patent Specification Nos. WO 94/13643, WO 94/13644, WO 94/13661, WO 94/13676 and WO 94/13677, which are hereby incorporated by reference.

Suitable atypical anti-depressants include: bupropion, lithium, nefazodone, trazodone and viloxazine, and pharmaceutically acceptable salts thereof.

Suitable classes of anti-anxiety agent include benzodiazepines and 5-HT_{1A} agonists or antagonists, especially 5-HT_{1A} partial agonists, and corticotropin releasing factor (CRF) antagonists.

Suitable benzodiazepines include: alprazolam, chlordiazepoxide, clonazepam, chlorazepate, diazepam, halazepam, lorazepam, oxazepam and prazepam, and pharmaceutically acceptable salts thereof.

Suitable 5-HT_{1A} receptor agonists or antagonists include, in particular, the 5-HT_{1A} receptor partial agonists buspirone, flesinoxan, gepirone and ipsapirone, and pharmaceutically acceptable salts thereof.

Therefore, in a further aspect of the present invention, there is provided a pharmaceutical composition comprising a compound of the present invention and an anti-depressant or anti-anxiety agent, together with at least one pharmaceutically acceptable carrier or excipient.

In a further or alternative aspect of the present invention, there is provided a product comprising a compound of the present invention and an anti-depressant or anti-anxiety agent as a combined preparation for simultaneous, separate or sequential use for the treatment or prevention of depression and/or anxiety.

In accordance with the method of the present invention, the individual components of the combinations of the present invention can be administered separately at different times during the course of therapy or concurrently in divided or single combination forms. The instant invention is therefore to be understood as embracing all such regimes of simultaneous or alternating treatment, and the term “administering” is to be interpreted accordingly.

For the treatment of the clinical conditions and diseases noted above, the compounds of this invention may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

Preferably the compositions containing an the ERβ selective agonists of use according to the present invention are in unit dosage forms such as tablets, pills, capsules, wafers and the like. Additionally, the ERβ selective agonists of use according to the present invention may be presented as granules or powders for extemporaneous formulation as volume defined solutions or suspensions. Alternatively, the ERβ selective agonists of use according to the present invention may be presented in ready-prepared volume defined solutions or suspensions. Preferred forms are tablets and capsules.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally include aqueous solutions, suitably flavored syrups, aqueous or oil suspensions, and flavored emulsions with edible oils such as cottonseed oil, sesame oil, coconut oil, peanut oil or soybean oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

Compositions of the present invention may also be administered via the buccal cavity using conventional technology, for example, absorption wafers.

Compositions in the form of tablets, pills, capsules or wafers for oral administration are particularly preferred.

The dosage regimen utilizing the compositions of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

In the treatment of a condition in accordance with the present invention, an appropriate dosage level of the ER beta agonist will generally be about 0.01 μg to 50 mg per kg patient body weight per day which may be administered in single or multiple doses. Preferably, the dosage level will be about 0.1 μg to about 25 mg/kg per day; more preferably about 0.5 μg to about 10 mg/kg per day. For example, for treating depression, a suitable dosage level is about 0.1 μg to 25 mg/kg per day, preferably about 0.5 μg to 10 mg/kg per day, and especially about 1 μg to 5 mg/kg per day. In larger mammals, for example humans, a typical indicated dose is about 300 μg to 400 mg orally. A compound may be administered on a regimen of several times per day, for example 1 to 4 times per day, preferably once or twice per day. When using an injectable formulation, a suitable dosage level is about 0.1 μg to 10 mg/kg per day, preferably about 0.5 μg to 5 mg/kg per day, and especially about 1 μg to 1 mg/kg per day. In larger mammals, for example humans, a typical indicated dose is about 100 μg to 100 mg i.v. A compound may be administered on a regimen of several times per day, for example 1 to 4 times per day, preferably once or twice per day.

Dosages of the active ingredients of the present invention, which can be combined with the estrogen receptor beta agonists of the present invention, can be administered in accordance with directions provided by the manufacturer or in publications describing the administration of said active ingredients. Oral dosages of the active ingredients of the present invention which can be combined with the estrogen receptor beta agonists of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day.

Pharmaceutical compositions of the present invention may be provided in a solid dosage formulation preferably comprising about 100 μg to 500 mg active ingredient, more preferably comprising about 100 μg to 250 mg active ingredient. The pharmaceutical composition is preferably provided in a solid dosage formulation comprising about 100 μg, 1 mg, 5 mg, 10 mg, 25 mg, 50 mg, 100 mg, 200 mg or 250 mg active ingredient.

The following examples are provided for the purpose of further illustration only and are not intended to be limitations on the disclosed invention.

It will be appreciated that the amount of the ERβ selective agonist required for use in the treatment or prevention of major depressive disorders will vary not only with the particular compounds or compositions selected but also with the route of administration, the nature of the condition being treated, and the age and condition of the patient, and will ultimately be at the discretion of the patient's physician or pharmacist.

As used herein, the term “depression” includes major depressive episodes, major depressive disorder and seasonal affective disorder.

As used herein, the term “major depressive disorder” includes single or recurrent major depressive episodes, with or without psychotic features, catatonic features, melancholic features, atypical features or postpartum onset and, in the case of recurrent episodes, with or without interepisode recovery and with or without seasonal pattern.

Other mood disorders encompassed within the term “major depressive disorder” include dysthymic disorder with early or late onset and with or without atypical features; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood.

Major depressive disorders may also result from a general medical condition including, but not limited to, myocardial infarction, diabetes, miscarriage or abortion, etc.

A “major depressive episode” is defined as at least two weeks of depressed mood or loss of interest, which may be accompanied by other symptoms of depression. The symptoms must persist for most of the day (i.e. for at least two thirds of the patients' waking hours), nearly every day (i.e. for at least ten out of fourteen days) for at least two consecutive weeks. A “depressed mood” is often described by the patient as feeling sad, hopeless, helpless or worthless. The patient may also appear sad to an observer, for example, through facial expression, posture, voice and tearfulness. In children and adolescents, the mood may be irritable. A “loss of interest” is often described by the patient as feeling less interested in hobbies or not feeling any enjoyment in activities that were previously considered to be pleasurable.

A major depressive episode may be accompanied by other symptoms of depression including significant weight loss when not dieting or weight gain (e.g. a change of more than 5% body weight in one month), or decrease or increase in appetite; insomnia or hypersomnia; psychomotor agitation or retardation; fatigue or loss of energy, feelings of worthlessness or excessive or inappropriate guilt; diminished ability to think or concentrate; or indecisiveness; and recurrent thoughts of death, recurrent suicidal ideation with or without a specific plan, or a suicide attempt.

As used herein, the term “perimenopausal depression” refers to depression occurring during the perimenopausal period. Perimenopause refers to the time when the ovarian response to pituitary gonadotropins (follicle-stimulating hormone [FSH] and luteinizing hormone [LH]) decreases, initially resulting in shorter follicular phases (thus, shorter menstrual cycles), fewer ovulations, decreased progesterone production, and more irregularity in cycles. Eventually, the follicle fails to respond and does not produce estrogen. Without estrogen feedback, circulating levels of LH and FSH rise substantially. Circulating levels of estrogens and progesterone are markedly reduced.

As used herein, the term “premenstrual syndrome” refers to a variety of symptoms that occur after ovulation and disappear after menstruation. These symptoms include breast tenderness, abdominal bloating, headache, weight gain and behavioral changes.

As used herein, the term “post-partum depression” refers to depression occurring during the post-partum period. Post partum refers to the first weeks following delivery.

As used herein, the term “manic depression” refers to alternate states of manic and major depressive feelings.

As used herein, the term “obsessive compulsive behavior” refers to recurrent, unwanted, intrusive ideas, images, or impulses that seem silly, weird, nasty, or horrible (obsessions) and by urges to do something that will lessen the discomfort due to the obsessions (compulsions).

As used herein, the term “seasonal affective disorder” refers to a type of depressive disorder which is a cyclic, seasonal condition in which the signs and symptoms of the disorder usually appear during the winter. Signs and symptoms of the disorder include depression, loss of energy, anxiety, irritability, headaches, increased sleep, loss of interest in sex, overeating (especially foods high in carbohydrates), weight gain, and difficulty concentrating and processing information. The patient is usually free of the symptoms during spring and summer, but some patients do have exacerbated symptoms of depression in the spring. Others may experience periods of mania or hypomaina, a less intense form of mania, during the summer. Characteristics of mania may include persistent elevated mood, hyperactivity, and inflated self-esteem.

As used herein, the term “mild cognitive impairment” refers to a condition where patients have sharp thinking and reasoning skills, but their short-term memory has declined. It could be a precursor to Alzheimer's disease.

As used herein, the term “attention deficit disorder” refers to Attention Deficit Hyperactivity Disorder, or ADHD, which is broken down into three different subtypes: Combined Type, Predominantly Inattentive Type, and Predominantly Hyperactive-Impulsive Type. ADHD is characterized by distractability, impulsivity and hyperactivity.

As used herein, the term “sleep disorders” includes disturbances of sleep that affect a subject's ability to fall and/or stay asleep, and involve sleeping too little, too much or resulting in abnormal behavior associated with sleep.

As used herein, the term “mood disorders” includes irritability, impulsivity and anger management issues.

As used herein, the term “multiple sclerosis” is the result of damage to myelin—a protective sheath surrounding nerve fibres of the central nervous system. It includes blurred vision, weak limbs, tingling sensations, unsteadiness, fatigue, chronic pain and rheumatoid arthritis.

As used herein, the term “Parkinson's disease” can be described as a chronic, progressive, neurologic disorder. An important part of its mechanism is the loss of the neurotransmitter dopamine in a group of brain structures that control movements. Its major manifestations are variable but can include hand tremor, slowness of movements, limb stiffness, and difficulties with gait and balance, chronic pain and rheumatoid arthritis.

As used herein, the term “inflammatory bowel disease (IBD)” can include disease of either or both of the large and small bowel. Ulcerative colitis and Crohn's disease are both forms of IBD. IBD can be active, which is characterized by acute inflammation. It can also be chronic, which is characterized by architectural changes of crypt distortion and scarring.

As used herein, the term “irritable bowel disease” refers to a functional gastrointestinal disorder which is characterized by gas, pain, bloating, nausea, vomiting, mucous in the stool, constipation and diarrhea.

As used herein, the term “treatment” refers both to the treatment and to the prevention or prophylactic therapy of the aforementioned conditions.

As used herein, the term “CNS-penetrating” refers to a compound that is active upon the central nervous system (CNS), such as the brain, following systemic administration.

As used herein, the term “orally active” refers to a compound which when given by mouth results in at least 5% of the dose being absorbed into the systemic circulation.

### EXAMPLE 1

Estrogen Receptor Binding Assay

The estrogen receptor ligand binding assays are designed as scintillation proximity assays employing the use of tritiated estradiol and recombinant expressed estrogen receptors. The full length recombinant human ER-α and ER-β proteins are produced in a bacculoviral expression system. ER-α or ER-β extracts are diluted 1:400 in phosphate buffered saline containing 6 mM α-monothiolglycerol. 200 μL aliquots of the diluted receptor preparation are added to each well of a 96-well Flashplate. Plates are covered with Saran Wrap and incubated at 4° C. overnight.

The following morning, a 20 μL aliquot of phosphate buffered saline containing 10% bovine serun albumin is added to each well of the 96 well plate and allowed to incubate at 4° C. for 2 hours. Then the plates are washed with 200 μL of buffer containing 20 mM Tris (pH 7.2), 1 mM EDTA, 10% Glycerol, 50 mM KCl, and 6 mM α-monothiolglycerol. To set up the assay in these receptor coated plates, add 178 μL of the same buffer to each well of the 96 well plate. Then add 20 μL of a 10 nM solution of ³H-estradiol to each well of the plate.

Test compounds are evaluated over a range of concentrations from 0.01 nM to 1000 nM. The test compound stock solutions should be made in 100% DMSO at 100× the final concentration desired for testing in the assay. The amount of DMSO in the test wells of the 96 well plate should not exceed 1%. The final addition to the assay plate is a 2 μL aliquot of the test compound which has been made up in 100% DMSO. Seal the plates and allow them to equilibrate at room temperature for 3 hours. Count the plates in a scintillation counter equipped for counting 96 well plates.

### EXAMPLE 2

Protocol: Murine Tryptophan Hydroxylase mRNA Expression. In situ Hybridization and Real Time Quantitative PCR methods.

Animals and treatment groups. Female mice (13-16 wks of age) are ovariectomized by the vendor (C57BL/6s from Charles River; E R Knockout animals from Taconic) and shipped to the Merck Research Laboratories one week later. Animals are fed a soy-free rodent chow upon arrival at the Merck animal facility, where they are given an additional week to adjust to the new environment. Mice are orally dosed in the morning (once daily for 4 days) with 0.2 cc of vehicle (20% ethanol:30% polyethylene glycol:50% water) or compound (0.1-30 mpk for dose response curves; 10 mpk for single-dose experiments); estradiol 17-beta is subcutaneously administered at 0.2 mpk in sesame oil (0.1 cc). Approximately six hours following the fourth dose, mice are deeply anesthetized with ketamine/xylazine, blood is collected via cardiac puncture, allowed to clot, and then serum is collected by centrifugation. The uterus is dissected out of the abdominal cavity, placed on a dissecting board, and fat is removed with a razor blade. The uterus is placed into a Microfuge tube containing 0.9% saline and placed at 4° C. overnight The next day, the uteri are removed from the saline, blotted on a napkin and weighed.

Brains intended for use in the in situ hybridization experiments are removed from the skull and immediately frozen on dry ice. For TPH-Taqman® RNA measurements, the brains are removed from the skull, placed ventral side up in a mouse brain block on ice, and ice-cold razor blades are inserted into the block at 1 mm intervals. The caudal extent of the hypothalamus is used as an anatomical marker for the placement of the first razor blade, and 4 blades are placed in sequential slots, caudally. The 4 sections are examined and the two that encompass the greatest extent of the dorsal raphe are placed in a Microfuge tube containing RNA-later and placed at 4° C., overnight.

In situ hybridization-TPH riboprobe. A 265 base TPH mRNA riboprobe, SEQ ID NO:1, below, was used to perform in situ hybridization on cryostat-cut 16 μm thick coronal dorsal raphe sections. The TPH cDNA sequence chosen was at the 5′ end of the gene, extending from nucleotide 239 to 503 (GenBank accession no. J04758). The antisense and sense probes were synthesized using ³⁵S-labeled UTP (NEN-Dupont, Boston, Ma.) incorporated into cRNA. The probe was transcribed using a cDNA template containing RNA polymerase sequence extensions for T7 (antisense) and T3 (sense). A Nuctrap push column was used for removal of unincorporated nucleotides (Stratagene, La Jolla, Calif.). The template was amplified from mouse brain cDNA (Clontech, Palo Alto, Calif.) using primers against the mouse sequence.

SEQ ID NO: 1:
TACACATCGA GTCCCGGAAA TCAAAGCAAA GAAATTCAGA
ATTTGAGATA TTTGTTGACT GCGACATCAG CCGAGAACAG
TTGAATGACA TCTTCCCCCT GCTGAAGTCG CACGCCACCG
TCCTCTCGGT GGACTCGCCC GATCAGCTCA CTGCGAAGGA
AGACGTTATG GAGACTGTCC CTTGGTTTCC AAAGAAGATT
TCTGACCTGG ACTTCTGCGC CAACAGAGTG CTGTTGTATG
GATCCGAACT TGACGCCGAC CACCC

In situ hybridization—Protocol and Evaluation. Slides containing 16 μm thick coronal dorsal raphe sections of the mouse brain were briefly post-fixed in 4% formaldehyde in 1× PBS buffer (Ambion, Austin, Tex.), rinsed in 1× PBS, acetylated with 0.2% acetic anhydride in 0.1M triethanolamine, and rinsed in 2× saline sodium citrate (SSC) (2×SSC: 0.3M NaCl, 0.03M sodium citrate). After rinses and ethanol dehydration, sections were hybridized overnight at 55° C. with 5×10⁴ c.p.m. probe/μl. The following morning, sections were washed in 2×SSC, ribonuclease A (RNase A) at 37° C. for 30 min, 2×SSC, and 0.1×SSC at 65° C. Sections were dehydrated using ethanol, and apposed to β-sensitive film (Biomax MR, NEN-Dupont) for 6 days at room temperature.

Autoradiographic images of midbrain sections were anatomically matched between animals, and densitometry was performed using a CCD video camera (Dage-MTI Inc., Michigan City, Ind.) fitted with Nikon lenses (Nikon Canada, Inc.), and the Scion Image Program. The average gray scale optical density (O.D.) reading was obtained by subtracting the background reading outside the region of interest from the O.D. of the dorsal raphe nucleus. Analysis was performed on 3 coronal sections, representing the rostral to caudal extent of the DRN, spanning ˜270 μm.

Real Time Quantitative PCR Measurement of TPH Message in Murine Dorsal Raphe—TaqMan®

Murine TPH TaqMan® primers and probe sequences. Murine TPH forward primer is named mTPH-874F, its corresponding sequence is: 5′-CAC AGT TCA GAT CCC CTC TAC ACT-3′ (SEQ ID NO: 2), and it spans nucleotides 874 to 897. The murine TPH reverse primer is named mTPH-962R, its corresponding sequence is: 5′-GCA AAA CTG GGT TCA GCC AA-3′ (SEQ ID NO: 3), and it spans nucleotides 943 to 962. The murine TPH probe is named mTPH-926T, its corresponding sequence is: 5′-AGG AGT TCA TGG CAG GTG TCT GGC TCT-3′ (SEQ ID NO: 4), and it spans nucleotides 900 to 926.

Murine TPH GenBank accession #J04758 was referenced to design these primers and probe therefore the nucleotide numbering is based on this sequence.

Isolation of total RNA from mouse raphe slices for Taqman® analysis. Samples are stored in RNALater at 4° C. overnight followed by removal of RNALater & storage at −80° C. until isolation of the total RNA (2 slices weigh 25-50 mg). Slices are removed from −80° C. and placed in 1.0 ml TRIzol Reagent in FastPrep® processing tubes. Slices are homogenized with one pass at setting 6 for 30 s in FastPrep® 120 homogenizer using green capped tubes with bead matrix followed by 20 s at setting 6 after all samples have been processed. Samples are set at RT for 5 min to allow for complete dissociation of nucleoprotein complexes followed by centrifugation of samples at 12,000×g for 5 min at 4° C. Homogenates are transferred to 1.5 ml microfuge tubes and 100 ul BCP (Bromo-3-chloropropane) is added, samples are vortexed for 15 sec. and set at RT for 23 min. Samples are centrifuged at 12,000×g for 15 min at 4° C. The aqueous layer is removed and placed in a new RNAse-free sterile 1.5 ml microfuge tube. 5 ul 5 mg/ml glycogen is added to each sample and samples are vortexed. 500 ul isopropanol is added to each sample, samples are vortexed for 15 sec., set at RT 10 min., followed by centrifugation at 12,000×g for 15 min at 4° C. Supernatants are decanted and pellets washed with 500 ul ice cold 75% ethanol. Samples are centrifuged at 12,000×g for 15 min at 4° C., ethanol decanted and pellets air dried for 10 min. Pellets are resuspended in 30 ul prewarmed RNASecure (60° C.), and samples are heated at 60° C. for 10 min. Samples are stored at −80° C. until DNAse treatment & cDNA synthesis.

DNase treatment and cDNA preparation with wt mouse raphe slice total RNA for Taqman® analysis DNase treatment using DNA-free kit (Ambion). 5 ug total RNA sample is aliquotted to each well of a 96 well plate. 1× DNase I solution is added to each sample (DNase I sol.: DNase I buffer, DNase I, H₂O). Reactions are mixed and incubated at 37° C. for 30 min. Reactions are inactivated by addition of DNase Inactivation reagent beads, mixed well at RT for 3 min. and centrifuged at 2500 RPM for 1 min at 4° C. (25 ul reactions are run and inactivated with 1/10 volume of inactivation reagent.)

Reverse Transcription using Applied Biosystems Reagents: 10 ul of DNase I-treated total RNA is added to 40 ul of 1× reverse transcription reaction mix (DEPC H₂O, RT buffer, MgCl2, dNTP mix, random hexamers, RNase inhibitor, and MultiScribe RT) and reactions are incubated at 25° C. for 10 min., 48° C. for 30 min. and 95° C. for 5 min. Reverse transcription is halted addition of EDTA. Samples are transferred to a storage plate and stored at −20° C.

TaqMan® analysis of raphe slice CDNA for determination of relative levels of murine TPH mRNA. 2.5 ul of cDNA is added to each well of a 96-well plate with 22.5 ul of TaqMan® reaction mix (1X Universal Master Mix (ABI), 300 nM mTPH-874F and 300 nM mTPH-962R primers, 200 nM mTPH-926T probe, 20 nM forward and reverse rRNA primers, and 100 nM rRNA probe). Samples are run on an ABI PRISM® 7700 Sequence Detection Instrument (Applied Biosystems, Foster City, Calif.) and collected data is analyzed using Merck Biometrics TaqManPlus program.

### EXAMPLE 3

Mouse Forced Swim Test

Male Swiss Webster mice (Bantin and Kingman, Hull, UK), weighing 20-25 g were housed in groups of nine with free access to food and water in a humidity and temperature controlled room. They were maintained on a 12 hour light/dark cycle with lights on a t 0700 hours and animals were allowed to acclimatise for at least three days prior to use. All procedure were carried out in accordance with the UK Animals (Scientific Procedures) Act (1986) and its associated guidelines.

Mice were tested by placing them in a glass cylinder (height=25 cm; diameter=10 cm) containing water (24-25 degrees Celsius) to a depth of 14 cm. The time that the animal spent trying to escape, immobile and moving around the tube (swimming) were recorded in 1 minute time bins for 5 minutes.

The ERβ selective agonists are dissolved in sesame oil. The test compounds are injected SC in a volume of 10 mL/kg 30 minutes before testing. Test compounds that reduce immobility in the forced swim test have potential in the treatment of depression.

### EXAMPLE 4

Resident Intruder Assay

Aggression is tested by placing an ovariectomized female rat (intruder) into a cage occupied by a female rat (resident) of the same size for a defined period of time. An observer scores various aspects of aggression (attack, attack-time, bites, on-top, on-top time, and piloerection) displayed by the resident towards the intruder. A composite aggression score is computed (Ho et al Neuropsychopharmacology 24:502, 2001). Animals are treated with a vehicle control or the test compound and the level of aggression is determined.

### EXAMPLE 5

Separation-Induced Vocalisation

Male and female guinea-pigs pups are housed in family groups with their mothers and littermates throughout the study. Experiments are commenced after weaning when the pups are 2 weeks old. Before entering an experiment, the pups are screened to ensure that a vigorous vocalisation response is reproducibly elicited following maternal separation. The pups are placed individually in an observation cage (55 cm×39 cm×19 cm) in a room physically isolated from the home cage for 15 minutes and the duration of vocalisation during this baseline period is recorded. Only animals which vocalise for longer than 5 minutes are employed for drug challenge studies (approximately 50% of available pups may fail to reach this criterion). On test days each pup receives an oral dose or an s.c. or i.p. injection of test compound or vehicle and is then immediately returned to the home cage with its mother and siblings for 30 to 60 minutes (or for up to 4 hours following an oral dose, dependent upon the oral pharmacokinetics of the test compound) before social isolation for 15 minutes as described above. The duration of vocalisation on drug treatment days is expressed as a percentage of the pre-treatment baseline value for each animal. The same subjects are retested once weekly for up to 6 weeks. Between 6 and 8 animals receive each test compound at each dose tested.

CNS-penetrant ERβ selective agonists of use in the present invention are also effective in the attenuation of separation-induced vocalisations by guinea-pig pups as hereinafter defined.

Essentially, a vocalisation response in guinea-pig pups is induced by isolation from their mothers and littermates, which response is attenuated when a CNS-penetrant ERβ selective agonist is administered subcutaneously 30 minutes prior to isolation, wherein vocalisations during the first 15 minutes of isolation are attenuated with an ID₅₀≦20 mg/kg, preferably with an ID₅₀≦10 mg/kg, and especially with an ID₅₀≦5 mg/kg.

In an alternative method, the ERβ selective agonist is administered orally, 2 hours prior to isolation, wherein vocalisations during the first 15 minutes of isolation are attenuated with an ID₅₀≦20 mg/kg, preferably with an ID₅₀≦10 mg/kg, and especially with an ID₅₀≦5 mg/kg.

While the invention has been described and illustrated with reference to certain particular embodiments thereof, those sidled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for treating a disorder selected from: depression, perimenopausal depression, postpartum depression, premenstrual syndrome, manic depression, anxiety, dementia, obsessive compulsive behavior, mild cognitive impairment, attention deficit disorder, a sleep disorder, irritability, impulsivity, anger management, multiple sclerosis and Parkinson's disease, in a mammal by increasing the transcription of TPH which comprises administering to the mammal a therapeutically effective amount of an ERβ selective agonist.

2. The method of claim 1 wherein the disorder is depression.

3. The method of claim 1 wherein the disorder is anxiety.

4. The method of claim 1 wherein the disorder is dementia.

5. The method of claim 1 wherein the disorder is multiple sclerosis.

6. The method of claim 1 wherein the disorder is Parkinson's disease.

7. The method of claim 1 wherein the ERβ selective agonist is an orally active ERβ selective agonist.

8. The method of claim 7 wherein the ERβ selective agonist is a CNS-penetrating estrogen receptor beta selective agonist.

9. A pharmaceutical composition comprising an estrogen receptor beta selective agonist and at least one other active ingredient selected from the group consisting of: norepinephrine reuptake inhibitors, selective serotonin reuptake inhibitors (SSRIs), monoamine oxidase inhibitors (MAOIs), reversible inhibitors of monoamine oxidase (RIMAs), melatonin agonists, serotonin and noradrenaline reuptake inhibitors (SNRIs), corticotropin releasing factor (CRF) antagonists, a-adrenoreceptor antagonists and atypical anti-depressants.

10. The pharmaceutical composition of claim 9 wherein the norepinephrine reuptake inhibitor is selected from the group consisting of amitriptyline, clomipramine, doxepin, imipramine and trimipramine, amoxapine, desipramine, maprotiline, nortriptyline and protriptyline, and pharmaceutically acceptable salts and mixtures thereof.

11. The pharmaceutical composition of claim 9 wherein the selective serotonin reuptake inhibitor is selected from the group consisting of fluoxetine, fluvoxamine, paroxetine and sertraline, and pharmaceutically acceptable salts and mixtures thereof.

12. The pharmaceutical composition of claim 9 wherein the monoamine oxidase inhibitor is selected from the group consisting of isocarboxazid, phenelzine, tranylcypromine, selegiline, and the pharmaceutically acceptable salts and mixtures thereof.

13. The pharmaceutical composition of claim 9 wherein the reversible inhibitor of monoamine oxidase is selected from the group consisting of moclobemide and the pharmaceutically acceptable salts thereof.

14. The pharmaceutical composition of claim 9 wherein the serotonin and noradrenaline reuptake inhibitor is selected from the group consisting of venlafaxine and pharmaceutically acceptable salts thereof.

15. The pharmaceutical composition of claim 9 wherein the atypical anti-depressant is selected from the group consisting of bupropion, lithium, nefazodone, trazodone, viloxazine, and the pharmaceutically acceptable salts and mixtures thereof.

16. The method of claim 1 which also comprises one or more agents selected from a bisphosphonate, an estrogen, a selective estrogen receptor modulator, an androgen receptor modulator, an integrin antagonist, a cathepsin K inhibitor, an inhibitor of osteoclast proton ATPase, parathyroid hormone, calcitonin, Vitamin D, a synthetic Vitamin D analogue, and the pharmaceutically acceptable salts and mixtures thereof.
